(19) United States
(12) Patent Application Publication
(10) Pub. No.: US 2003/0124159 A1
(43) Pub. Date: Jul. 3, 2003

(54) Enhancing epidermal barrier development in skin

(75) Inventors: Gail Jenkins, Bedford (GB); Joanne Read, Rushden (GB); Katherine Louise Rumary, Bedford (GB)

Correspondence Address: UNILEVER, PATENT DEPARTMENT, 45 RIVER ROAD, EDGEWATER, NJ, 07020, US

(73) Assignee: Unilever Home & Personal Care USA, Division of Conopco, Inc

(21) Appl. No.: 10/262,744

(22) Filed: Oct. 2, 2002

(30) Foreign Application Priority Data
Oct. 4, 2001 (EP) 01308487.6

(51) Int. Cl. A61K 31/56; A61K 7/00

(52) U.S. Cl. 424/401; 514/179

(57) ABSTRACT

The invention relates to the field of skin compsitions and to the identification of novel effects of molecules when incorporated into a skin composition. More particularly the invention relates to systemic or topical compositions and their use in providing a variety of skin care benefits by enhancing the development of a healthy epidermal barrier layer in the skin through the activation of the nuclear receptor LXRα.

### FIELD OF THE INVENTION

The invention relates to the field of topical or systemic compositions and to the identification of novel effects for molecules when incorporated into such a composition. More particularly the ivention relates to these compositions and their use in providing a variety of skin care benefits by enhancing the development of a healthy epidermal barrier layer in the skin.

### BACKGROUND TO THE INVENTION

The epidermis is a stratifying keratinising epithelium, with its uppermost layer, the stratum corneum, providing the skin with structural integrity and a barrier to excess water loss. Keratinocytes provide the basal layer of cells in the epithelium which proliferate and differentiate as they migrate towards to the uppermost layer of the skin where they form the corneocytes of the stratum corneum. The stratum corneum provides a cornified envelope which also comprises specialised membrane complexes in spaces between the corneocytes which are derived from lipids synthesised with the epidermis and are required to maintain a permeability barrier.

The liver-X receptor (LXR) is a nuclear receptor to be present in human keratinocytes where it plays an integral role in the regulation of cell proliferation and differentiation as well as lipid metabolism within the epidermis. It is known in the art that 22-R hydroxycholesterol induces coordinate expression of the differentiation-specific genes encoding involucrin and transglutaminase 1, increase the information of the cornified envelope, and inhibit cellular proliferation (Hanley et al., Journal of investigative Dermatology, Vol 114 No. 3p. 545-553)

WO 98/32444 relates to the problem of epidermal barrier dysfuntion and discloses a specific sub-set of oxysterois that can be used to enhance barrier development via the activation of LXRα. This document importantly notes that structurally very similar oxysterol compounds and even cholesterol itself are not effective activators of LXRα. This highlights to the person skilled in the art that the LXRα is highly specific receptor.

A skin composition comprising guggul sterone in combination with a number of other active ingredients is known for the treatment of cellulite (U.S. Pat. No. 6,120,779). Cellulite arises from increased deposition of fat in adipocytes i.e. fat cells which lie underneath the dermis and provides a totally distinct technical problem from the need to maintain a permeability barrier in the epidermis.

Being able to improve epidermal barrier function is particularly advantageous when skin is dry or damaged, wherein any improvement in the barrier will reduce moisture loss and generally enhance the quality and flexibility of the skin.

The objective technical problem to be solved by the present ivention is to find alternative molecules capable of improving epidermal barrier development in the skin via the activation of the highly specific LXRα.

The prior art of WO 98/32444 teaches that the capacity to activate LXRα is restricted to a small group of oxysterols, it is therefore surprising to now find further molecules capable of activating LXRα and thus able to improve epidermal barrier properties in the skin.

### SUMMARY OF THE INVENTION

The present invention accordingly provides a group of compounds which have the newly identified ability to activate LXRα and thereby provide a means of improving epidermal barrier properties in the skin.

It is a first object of the invention to provide the use of a LXRα activating compound according to the general formulae;

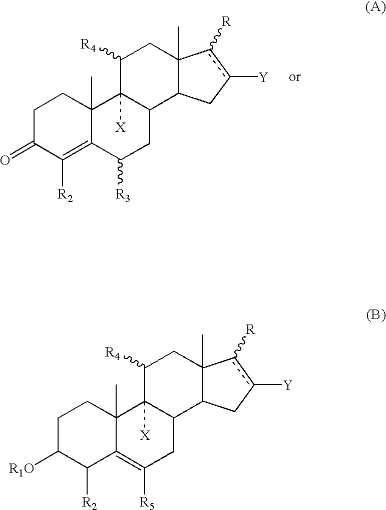

wherein;

R represents a hydrogen, a hydroxyl, a keto, an acetyl, a C₁ to C₇, substituted or unsubstituted, branched or unbranched, saturated or unsaturated alkyl group, or a substituted or unsubstituted, branched or unbranched usaturated C₈ alkyl group;

R₁ represents a lower alkyl group, a hydrogen or COR₆;

R₂ represents a hydrogen, a halogen or a hydroxyl group;

R₃ represents a hydrogen, a hydroxyl, a halogen, a keto or a lower alkyl group;

R₄ represents a hydrogen, a hydroxyl, or a keto group;

R₅ represents a hydrogen, a halogen, a hydroxyl or lower a alkyl group;

R₆ represents a lower alkyl group.

X represents a hydrogen, a methyl or a halogen;

Y represents a hydrogen, a hydroxyl, a acetyl or a keto group;

in the manufacture of a topical or systemic composition for enhancing epidermal barrier function of skin.

A systemic or topical composition comprising a compound of formula (A) or (B) optionally with one or more other ingredients, is also within the ambit of the present invention.

It is a second object of the invention to provide a cosmetic method of providing at least one skin care benefit selected from the group consisting of; treating/preventing dry skin; soothing irritated, red and/or sensitive skin; boosting/maintaining involucrin levels; reducing the rate of ageing; the method comprising applying to the skin a topical composition or dosing systemically, a systemic composition comprising a compound according to the formulae (A) or (B) as hereinbefore defined.

It is a third object of the invention to provide a systemic composition for enhancing epidermal barrier function of skin, said composition comprising an LXRα activating compound according to either formula above and a dermatologically acceptable vehicle, wherein the R represents —H, —OH, ═O, —COCH₃, —COHCH₃, ═CHCH₂OH, or —OCOCH₃.

It is a fourth object of the invention to provide a topical composition for enhancing epidermal barrier function of skin, said composition comprising an LXRα activating compound according either formula above and a pharmacologically acceptable carrier therefor, wherein the R represents —H, —OH, ═O, —COCH₃, —COHCH₃, ═CHCH₂OH, or —OCOCH₃.

### DETAILED DESCRIPTION OF THE INVENTION

Epidermal barrier function is determined by growth and differentiation of those cells within the skin epidermis associated with the development of the healthy comified epithelium required to maintain a permeability barrier.

The improvement of epidermal barrier function has been measured herein by two means; firstly by way of a reporter gene assay for activation of LXRα and secondly by the level of fillagrin expression detected in cells treated according to the invention. Filaggrin is well recognised as a marker of epidermal differentiation wherein an increase in filaggrin is indicative of enhanced barrier function within the skin by the development of a cornified epithelium (Komuves L G. et al., 1999 Journal of Investigative Dermatology 112:203-9.

### EXPLANATION OF THE FIGURES

FIG. 1: illustrates the reporter gene activity associated with cis-guggalsterone (cis-4,17(20)-pregnadien-3,16-diol) activation of LXRα.

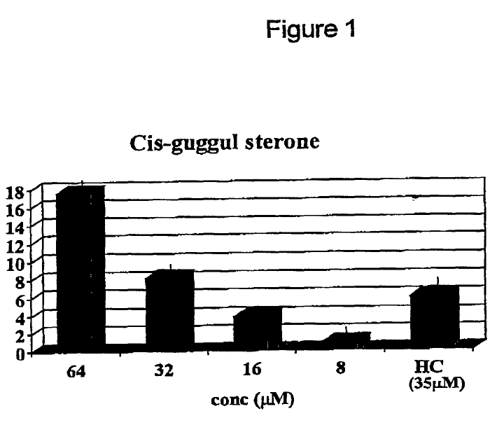

FIG. 2: illustrates the reporter gene activity associated with demosterol (cholesta-5,24-dien-3β-ol) activation of LXRα.

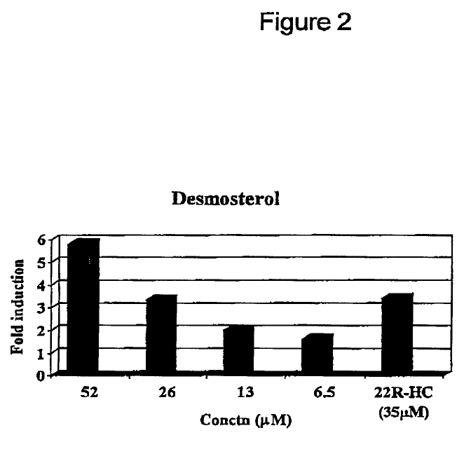

FIG. 3: shows molecular modeling of molecules which have been tested and shown to activate LXRα.

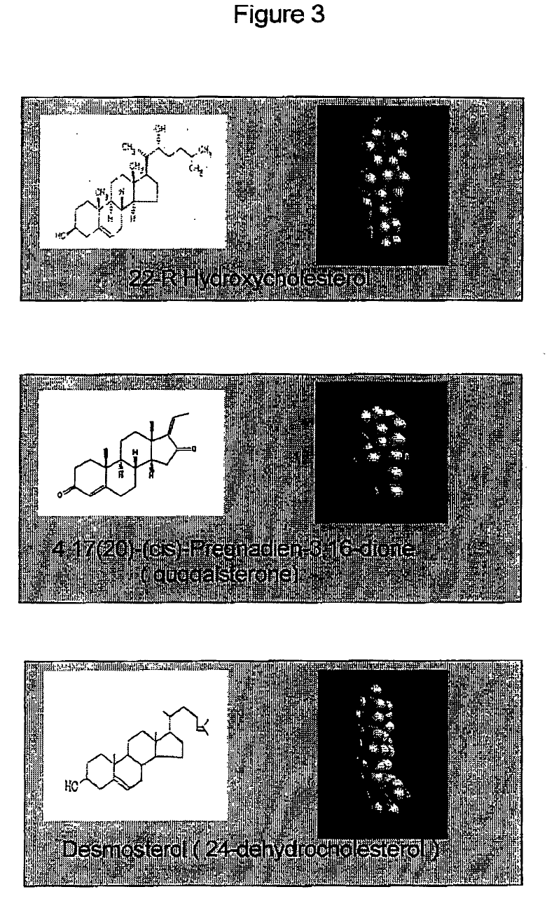

FIG. 4: shows molecular modeling of molecules which have been shown not to activate LXRα.

FIG. 5: illustrates the plasmid map for pNFkB-Luc.

FIG. 6: illustrates the conventional carbon numbering system for cholesterol-type molecules.

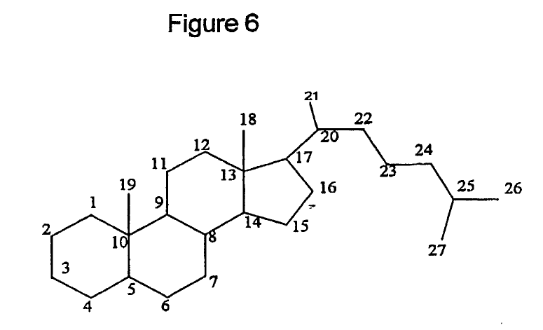

Contrary to conventionally accepted knowledge it has been found that the group of molecules capable of activating LXRα is not restricted to a small set of oxysterols as proposed in the prior art. Furthermore in one aspect of the invention it has been demonstrated that molecules which form part of the group identified herein have a greater ability to activate LXRα than those oxysterols identified in the prior art and thereby provides more effective agents for enhancing barrier properties of the epidermis.

The bond by which the R group is linked to the carbon at position 17 will depend on the nature of the R group (indicated by wavy bond). Where R is a hydrogen or a hydroxyl group or acetyl group the bond will be saturated, whereas when R is a keto group the bond will be unsaturated. When R is an alkyl group this group may be linked to the carbon at position 17 via a saturated or unsaturated bond, preferably this is an unsaturated bond.

For the purpose of the present invention R may represent a hydroxyl, a keto or an acetyl group.

R may also represent a C₁ to C₇ (i.e. including C₁,C₂,C₃,C₄,C₅,C₆ and C₇) substituted or unsubstituted, saturated or unsaturated, branched or unbranched alkyl group. Preferably said C₁ to C₇ alkyl group comprises at least one substituted group selected from hydroxyl, keto and acetyl groups and R may in particular represent substituted alkyl groups having two and three of said substitutions. More preferably the alkyl groups have undergone substitution with one or more keto or hydroxyl groups. Further preferred an alkyl R group is substituted at one or more positions corresponding or equivalent to C₂₀, C₂₁, C₂₂ and C₂₃ shown in FIG. 7. Where the substitution is with a keto group this is most preferably bonded to C₂₀, whereas when substitution is with a hydroxyl group this is most preferably bonded to a carbon at C₂₁ and or C₂₂.

It is preferred that the alkyl R group remains unbranched as this helps to maintain a favoured linear configuration, however in the event that the alkyl group is branch said branches preferably comprise 2 carbons, more preferably 1 carbon.

Where the R group is an alkyl group as described above this will preferably have some degree of unsaturation. Preferably unsaturation occurs in the form of one or more substituted keto groups.

Where R represents an unsaturated C₁ to C₆ alkyl group it is most preferred that this group has the formula —C(CH₃)(CH₂)₂C═C(CH₃)₂.

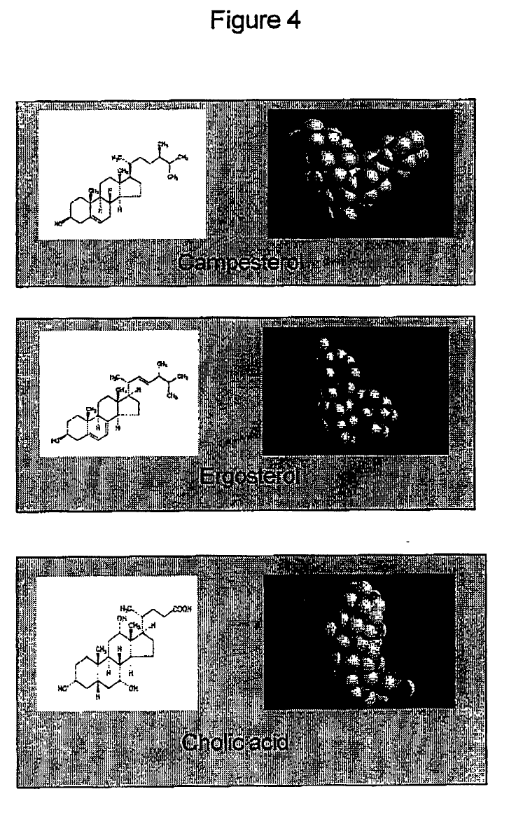

While not wishing to be bound by any theory the applicants believe that it is the conformation of the R group of a molecule according to the general formulae provided herein which determines the correct interaction with the active site and thereby the activation of LXRα. More particularly from computer modeling of molecular structures it is believed, where the R group is a carbon chain, that in order to allow correct active site interaction the R group should preferably adopt a substantially linear conformation as illustrated in FIG. 4. This may be achieved in those molecules wherein the R group is a substantially linear carbon chain and/or has at least one unsaturated C—C bond.

It is also believed that the most effective activators of LXRα comprise a small R group. In a preferred embodiment the R group of the LXRα activating compound therefore represents a hydrogen, a hydroxyl, a keto or an unsubstituted or, more preferably, substituted C₁ to C₄ alkyl group. Preferably substitution occurs at C₂₀ or C₂₁ within the alkyl group. Where the R group is an alkyl group it is preferred that this is forms an unsaturated bond with C₁₇ of the ring structure.

In a preferred embodiment R represents a hydrogen, a hydroxyl, a keto or a substituted/unsubstituted C₁ to C₄ alkyl group. Suitable unsubstituted groups include methyl, ethyl, n-propyl; isopropyl, n-butyl, isobutyl or ter-butyl.

In a most preferred embodiment R is selected from the group consisting of —H, —OH, ═O, —COCH₃, —COHCH₃, ═CHCH₃, ═CHCH₂OH, —OCOCH₃ and C(CH₃)(CH₂)₂C═C(CH₃)₂.

A “lower alkyl” as employed herein includes both straight and branched chain radicals of up to four carbon atoms, examples of suitable groups are outlined above. In a preferred embodiment R₁ is a hydrogen.

R₂ represents a hydrogen, a halogen preferably chlorine or a hydroxyl group, preferably R₂ represents a hydrogen.

R₃ represents a hydrogen, a halogen preferably a fluorine or chlorine, a keto or a lower alkyl group. Preferably R₃ is either a keto group or a hydrogen. In a most preferred embodiment R₃ is a hydrogen.

Preferably R₄ and R₅ represent a hydroxyl group or hydrogen, most preferably these represent a hydrogen.

R₆ represents a lower alkyl, preferably a methyl group.

X preferably represents a hydrogen, a fluorine or a chlorine, most preferably X is a hydrogen.

Preferably Y represents a hydrogen, a hydroxyl or a keto group.

When Y is a hydrogen, in a compound according to general formula A, a double bond may form between C₁₆ and C₁₇.

In a compound according to formula B, when Y is hydrogen R₁ is a preferably hydrogen or —COR₆. Preferably when Y is a keto group the activating molecule conforms to general formula A, whereas, when Y is a hydroxyl group the activating molecule preferably conforms to general formula B.

In a most preferred embodiment the activating compound conforms to formula A wherein Y is a keto group.

Where R is a hydrogen or a hydroxyl group, Y is preferably a keto group in an activating compound according to formula A.

Where R is —COCH₃, Y is preferably a hydrogen or a keto group in a activating compound according to either A or B, preferably according to formula A.

Where R is ═CHCH₃ or —OCOCH₃, Y is most preferably a keto group in an activating compound according to general formula A.

Where R is ═CHCH₂OH, Y is preferably either, a hydrogen in an activating compound according to general formula A, wherein R₄ is preferably a hydroxyl group; or a hydroxyl group in an activating compound according to formula B wherein R₁ is a hydrogen.

Where R is C(CH₃)(CH₂)₂C═C(CH₃)₂, Y is preferably a hydrogen in an activating compound according to formula B wherein R₁ is also a hydrogen.

In a preferred embodiment of the use according to the invention the desired activation of LXRα is provided by a compound selected from the group consisting of; 4-androsten-3,16dione, 4-androsten-3,16-dione, androst-3,6,16-trione, 4-androsten-17beta-ol-3, 16-dione acetate, 16-ketotesterone, 3β-acetoxypregna-5,16-dien-20-one, 3β-acetoxypregna-5-en-20-one, 3β-hydroxypregna-5,16-dien-20-one, 3β-hydroxypregna-5-en-20-one, 5,16-dien-pregnane-3,20-diol, 4,16-dienpregna-3,20-dione, 4,17(20)-(cis)-pregnadien-3,16dione, 4,17)(20)-(trans)-pregnadien-3,16-dione, 4-pregnen-3,16,20-trione, 4,17)(20)-pregnadien-11beta,21-diol-3-one, 5,17(20)-pregnadien-3,16-diol-diacetate, 5,17(20)-pregnadien-3,16-diol, 5-pregnen-3beta,16alpha,21-triol-20-one, 24hydroxychol-4-en-3-one, cholesta-5,24-dien-3β-ol, cis-guggal sterone and desmosterol, and mixtures thereof.

In a most preferred embodiment the invention relates to the use of 4,17(20)-(cis)-pregnadien-3,16-dione in the manufacture of a composition for enhancing epidermal barrier function of the skin.

The preparation of all compounds the compound has been described in the literature and/or are commercially available e.g. from Sigma Chemical Company.

In providing the composition of the invention an effective amount of an LXRα activator molecule capable of bringing about a detectable increase in the level of reporter gene expression and which will accordingly improve the barrier development of the skin in incorporated therein. The amount of LXRα activator molecule, or a mixture thereof, present in the final composition according to the invention will typically be from 0.001 to 50% wt, preferably from 0.01 to 10% weight, and most preferably from 0.1 to 1% weight of said composition. Typically the concentration of the activator molecule will be approximately 1 to 10 μM.

In a preferred embodiment of the invention a topical composition for enhancing epidermal barrier function of skin comprises,

(a an effective amount of an activator of LXRα according selected from the group consisting of; 4-androsten-3,16-dione, 4-androsten-3,16-dione, androst-4-ene-3,6,16-trione, 4-androsten-17beta-ol-3,16-dione acetate, 16-ketoteatosterone, 3β-acetoxypregna-5,16-dien-20-one, 3β-acetoxypregna-5-en-20-one, 3β-hyroxypregna-5,16-dien-20-one, 3β-hydroxypregna-5-en-20-one, 5,16-dien-pregnane-3,20-diol, 4,16-dienpregna-3,20-dione, 4-pregnen-3,16,20-trione, 4,17(20)-pregnadien-11beta,21-diol-3-one, 5,17(20)-pregnadien-3,16-diol-diacetate, 5,17(20)-pregnadien-3,16-diol, 5-pregnen-3beta, 16alpha,21-triol-20-one, 24-hydroxychol-4-en-3-one, cholesta-5,24-dien-3β-ol, stigmasta-5,22-dien-3β-ol, cis-guggal sterone and desmosterol, and mixtures thereof, and

(b a dermatologically acceptable vehicle.

Another preferred embodiment of the invention provides a systemic composition for enhancing epidermal barrier function of skin, said composition comprising;

(a) an LXRα activating compound selected from the group consisting of, 4-androsten-3,16-dione, 4-androsten-3,16-dione, androst-4-ene-3,6,16-trione, 4-androsten-17beta-ol-3,16-dione acetate, 16-ketotestosterone, 3β-acetoxypregna-5,16-dien-20-one, 3β-acetoxypregna-5-en-20-one, 3β-hydoxypregna-5,16-dien-20-one, 3β-hydroxypregna-5-en-20-one, 5,16-dien-pregnane-3,20-diol, 4,16-dienpregna-3,20-dione, 4pregnen-3,16,20-trione, 4,17(20)-pregnadien-11beta,21-diol-3-one, 5,17(20)-pregnadien-3,16-diol-diacetate, 5,17(20)-pragnadien-3,16-diol, 5-pregnen-3beta, 16alpha,21-triol-20-one, 24-hydroxychol-4-en-3-one, cholesta-5,24-dien-3β-ol, stigmasta-5,22-dien-3β-ol, cis-guggal sterone and desmosterol, and mixtures thereof; and

(b a dermatologically acceptable vehicle.

A dermatologically acceptable vehicle acts as a dilutant, dispersant or carrier for the newly identified activators of LXRα in the composition, so as to facilitate its distribution when the composition is topically applied.

Dermatologically acceptable vehicles other than water can include liquid or solid emollients, solvents, humectants, thickeners and powders. Examples of each of these types of vehicle which can be used singly or as mixtures of one or more vehicles, are as follows:

Emollients, such as stearyl alcohol, glycerol monoricinoleate, glycerol monostearate, mink oil, cetyl alcohol, isopropyl isostearate, stearic acid, isobutyl palmitate, isocetyl stearate, oleyl alcohol, isopropyl luarate, hexyl laurate, decyl oleate, octadecan-2-ol, isocetyl alcohol, eicosanylalcohol, behenyl alcohol, cetyl palmitate, silicone oils such as dimethylpolysiloxane, di-n-butyl sebacate isopropyl myristate, isopropyl palmitate, isopropyl stearate, butyl stearate, polyethylene glycol, triethylene glycol, lanolin, cocoa butter, corn oil, cotton seed oil, tallow, lard, olive oil, palm kernal oil, rapeseed oil, safflower seed oil, evening primrose oil, soybean oil, sunflower seed oil, avocado oil, olive oil, sesame seed oil, coconut oil, arachis oil, castor oil, acetylated lanolin alcohols, petroleum jelly, mineral oil, butyl myristate, isostearic acid, palmitic acid, isopropyl linoleate, lauryl lactate, myristyl lactate, decyl oleate, myristyl myristate;

Propellants such as tichlorofluoromethane, dichlorodifluoro- methane, dichlorotetrafluoroethane, monochlorodifluoromethane, trichlorotrifluoroethane, propane, butane isobutanem demethyl ether, carbon dioxide, nitrous oxide;

Solvents such as ethyl alcohol, methylene chloride, isopropanol, acetone, ethylene glycol monoethyl ether, diethlyene glycol monobutyl ether, diethylene glycol monoethyl ether, dimethyl sulphoxide, dimethyl formamide, tetrahydrofuran;

Powders, such as chalk, talc, fullers earth, kaolin, starch, gums, colloidal silica sodium polacrylate, tetre alkyl and/or trialkyl aryl ammonium smectites, chemically modified magnesium aluminium silicate, organically modified montmorillonite clay, hydrated aluminium silicate, fumed silica, carboxyvinyl polmer, sodium carboxymethyl cellulose, ethylene glycol monostearate.

The dermatologically acceptable vehicle will usually form from 10 to 99.99% wt, preferably from 50 to 99% of the final composition ready for use by the consumer.

The composition may also comprise water, usually up to 98% volume, preferably 5 to 80% volume of said final composition.

The composition according to the invention which is primarily intended as a product for topical application to the human skin, especially as an agent for reducing the permeability to water of the skin, particularly when the skin is dry or damaged, in order to reduce moisture loss and generally enhance the quality and flexibility of the skin. Enhancing epidermal barrier function thus allows a person to gain from a number of cosmetic skin care benefits. Accordingly an embodiment comprises a cosmetic method of providing at least one skin care benefit selected from the group consisting of; treating/preventing dry skin; soothing irritated, red and/or sensitive skin; boosting/maintaining involucrin levels; the method comprising applying to the skin a topical composition described above.

The skin composition of the invention can be formulated as a lotion having a viscosity of from 4,000 to 10,000 mPas, a fluid cream having a viscosity of from 10,000 to 20,000 mPas or a cream having a viscosity of from 20,000 to 100,000 mPas or above at a temperature of 20° C. The composition may be packaged in a container to suit its viscosity and intended use by the consumer. For example a lotion or fluid cream can be packaged in a bottle or a roll-ball applicator or a propellant driven aerosol device or a container fitted with a pump suitable for finger operation. When the composition is a cream, it can simply be stored in a non-deformable bottle or a squeeze container, such as a tub or a lidded jar.

A composition according to the present invention for systemic administration may for example be adapted for oral administration, e.g. in the form of a tablet, lozenge, capsule, liquid (e.g. syrup or linctus) or as an injection (e.g. subcutaneous or intramuscular) or infusion or as a suppository. Typical such formulation techniques and appropriate pharmacologically acceptable carriers are well known to those skilled in the art. Suitable compositions for oral administration include those adapted for delayed release and/or for release in the lower gastrointestinal tract.

Another means of systemic dosing comprises dosing any of the aforementioned compositions in a food product which therefore does not necessarily require use of a pharmacologically acceptable carrier.

The invention accordingly also provides a closed container containing a cosmetically acceptable composition as herein defined.

### EXAMPLE 1

Reporter Gene Assay

The activation of LXRα has been determined by a reporter gene assay based on that described by Kliewer et al (Nature 358 771-774 1992). Wherein cos-7 cells (ECACC No. 87021302) were seeded in 24-well plates at a density of 5×10⁴ cells/well. Cells were grown overnight at 37° C./5% CO₂ in DMEM containing 10% FCS, 2mM L-glutamine, 100 ul/ml penicillin and 100 g/ml streptomycin.

Generation of Reporter Gene Constructs

A commerically available vector-pNFκB-Luc (Clontech) was used as the basic reporter plasmid as it contained the firefly luciferase gene downstream of the thymidine kinase promoter element. The NFκB consensus sequence was exised using restriction enzymes Mlu I and Bgl II and replaced with 3 direct repeats of the DNA response element sequence for the LXR nuclear receptor.

Generation of LXR Response Element:

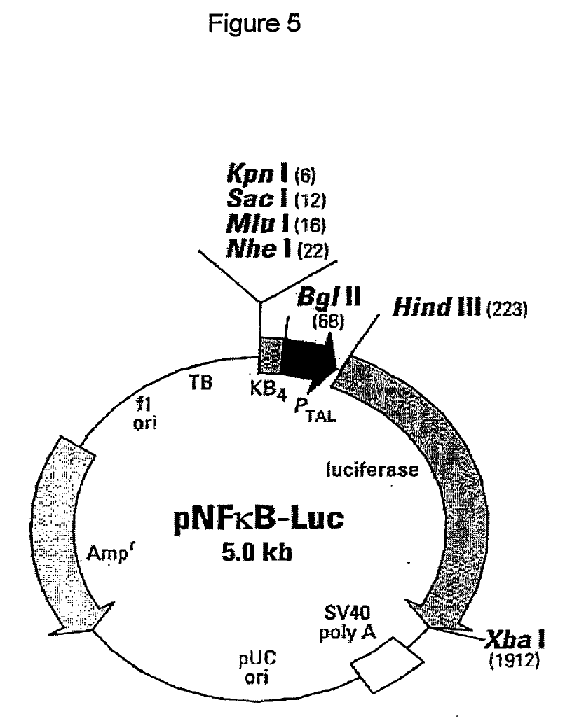

The response element was taken from Willy, P. et al (1995)(from a promoter region of the mouse mammary tumour virus), and repeated three times and encorporated restriction enzyme sites for Mlu I and Bgl II at either end to orientate the fragment during cloning (FIG. 5). This long oligonucleotide was synthesised, and an annealing primer designed to allow production of a double-stranded DNA template by Klenow fill-in.

This dsDNA template along with the vector pNFκB-Luc were then cut with both Mlu I and Bgl II restriction enzymes to allow cloning of the insert into this vector. ligation of the insert and vector occurred, followed by heat-shock transformation into a E. coli strain (JM109) followed by selection of recombinants on LB agar+Ampicillin (100 μg/ml). Mini liquid cultures of each colony generated were established and mini-plasmid preparations done (Qiagen protocols followed), furthered by restriction digests to check the size of the recombinant insert. These vectors were checked finally by DNA sequencing to prove they contained the LXR response element sequence in the correct orientation.

LXR response element;

(SEQ ID NO.1)

5′GGTTTA aata AGTTCA 3′
LXR response element oligo;
Mlu I site

(SEQ ID No.2)

5′gcatt cacgcgt ccag GGTTTA aata AGTTCA gttcacag
GGTTTA aata AGTTCA gttcacag GGTTTA aata AGTTCA
ggcaac agatct tacgcatg 3′
BgI II site
LXR response element annealing primer;

(SEQ ID NO.3)

5′catgcgtaagatctgttgcc 3′

Generation of RXRα Expression Vector:

The pRSV/hRXRα was prepared via the method of Collingwood T N et al. 1997. J Biol Chem. 272: 13060-5. Transformation was performed as described above, and bulk plasmid preparations were performed from 100 ml overnight cultures. The selectable antibiotic for this vector was 100 μg/ml Ampicilin.

Transfection of cells was performed using Lipofectamine (Gibco Brl) as directed by the manufactures. Transfected cells were incubated for 5 h at 37° C./5% CO₂ and serum then added to a final concentration of 2%. Cells were then incubated for a further 24 hours in the presence or absence of ligand. After 24 hours cell lysates were prepared and the level of firefly and renilla luciferase determined using the Dual luciferase assay system (Promega) and a MLX microtitre plate luminometer (Dynex).

Cells were washed with transfection media (DMEM) then transiently transfected with 4 plasmids: a LXR-responsive firefly luciferase reporter gene (pLXRE-luc); mammalian expression plasmids (pcDNA3.1/LXR, and pRSV/hRXRα) containing human LXR and RXRα cDNAs respectively and a control plasmid (pRLTK, Promega) which constitutatively expresses the renilla luciferase gene.

Transfected cells were incubated for 5 h at 37°C./5% CO₂ and serum then added to a final concentration of 2%. Cells were then incubated for further 24 hours in the presence or absence of ligand. After 24 hours cell lysates were prepared and the level of firefly and renilla luciferase determined using the Dual luciferase assay system (Promega) and a MLX microtitre plate luminometer (Dynex). The level of firefly luciferase (normalised against the renilla luciferase control) provides a measure of reporter gene activity. This in turn reflects the level of LXR activation

The level of firefly luciferase (normalised against the renilla luciferase control) provides a measure of reporter gene activity which in turn reflects the level of LXRα activation.

TABLE 1
Effect of agents on activation of LXR.

| | Reporter Gene | |
|---|---|---|
| | Activity | |
| | Foloinduction | |
| | relative to | |
| Extract | vertical Mean | Standard error |
| Cis-guggal Sterone | | |
| 16 μM | 3.9 | 0.16 |
| 32 μM | 8 | 0.6 |
| 64 μM | 17.3 | 0.78 |
| 22R-hydroxycholesterol (35 μM) | 6 | 2.0 |
| 22R-hydroxycholesterol (50 μM) | 6.59 | 1.8 |
| Desmosterol | | |
| 26 μM | 9.27 | 0.55 |
| 22R-hydroxycholesterol (32 μM) | 4.8 | 1.9 |

22R-hydroxcholesterol was present at the stated concentration in each of the positive control experiments.

The presence of active ligand stimulates reporter gene activity in a dose-dependent manner. Reporter gene expression is controlled by LXR and therefore reflects the level of LXR activation. Hence these data show that the agents claimed are acting as activators of LXR.

### Example 2

RNA Expression Analysis

Commercially available human epidermal cultures were obtained from Skin Ethic™. The cultures were incubated in DMEM supplemented with guggul sterone (conctri) or vehicle alone (X % ethanol) for X days. Medium was replaced every day.

RNA was then extracted from cultures using the Qaigen RNEasy™ minikit according to manufacturers' instructions. The RNA was than DNAse treated and quantified by measuring OD at 260 & 280 nm with a spectrophotometer.

The level of gene expression was then determined using the Integridenm array from Research Genetics. RNA expression in guggul sterone treated cultures was compared to that in cultures treated with vehicle alone according to manufacturers' instructions broadly detailed below.

(i) Preparing and Prehybridising Membranes

To prehybridise, each membrane was placed in a seperate roller bottle with the DNA spotted side facing inwards 5.0 ml MicroHyb (Research Genetics #HYB125.GF) was added to each bottle as well as the blocking agents;

(a) 5.0 μg human Cot-1 DNA (1 μg/ul, Life Technologies #15279-011) following denaturation by 99° C. for 6 mins and chilling on ice.

(b) 5.0 μg Poly dA (Research Genetics POLYA.GF, 1 μg/ul)

Prehybridisation was carried out at 47° C. for at least 2 h in a rotating oven.

(ii) Preparation of Labelled cDNA Probe

Annealing/Priming mRNA:

In a 0.5 ml PCR tube, the following were mixed:

1 ug total RNA in a total of 8 ul DEPC H2O

2.0 ul oligo dT (Research Genetics #POLYT.GF 10-20 mer 1 ug/ul)

After incubating at 7° C. for 10 mins, the tubes chilled on ice for 2 mins.

Elongation:

A pool of 20 mM ea dNTPs (excluding dATP),(Pharmacia #27-2035-02, 100 mM stocks), were made by mixing by 20 ul DEPC H2O, 10 ul each dCTP, dGTP, dTTP, (−2° C. storage).

A mastermix for 2 RNA samples was prepared by mixing;

14.4 μl 5× first strand buffer (Life Tech #18064-014)

2.4 μl DTT (0.1 M, Life Tech #18064-014)

3.6 μl pooled dCTP, dGTP, dTTP (see above)

3.6 μl reverse transcriptase (200 U/μl, Superscript II, Life Tech#18064-014)

2.4 μl 33 P dATP (Amersham BF1001-250Ci, 10 mCi/ml)

20 ul mastermix was added to each tube containing RNA and oligo dT, and the reaction incubated at 37° C. for 42° C. for 90 mins. Bio-Spin 6 columns (Bio-Rad #732-6002) were then used to remove unincorporated nucleotides according to the manufacturer's instructions.

A 2 ul sample was taken to check for activity by scintillation counting (32 P channel) and the total activity was the calculated for the fraction (˜100 μl) from each sample.

(iii) Hybridisation

The cDNA was denatured by heating to 99° C. for 3 mins and chilled on ice for 2 mins. Hybridisation was carried out at 42° C. for 16-20 hrs in a rotating oven.

(iv) Washing Membranes

Membranes were washed 4× with ˜30 ml wash 1 solution (2× SSC, 1% SDS) at 50° C. for 20 mins/wash in rotating oven. This was followed by 2 further washes in ˜30 ml wash 2 solution (0.5× SSC, 1% SDS) at 50° C. for 15 mins/wash in a rotating oven.

(v) Development of Phosphorimage

Images of the membranes were obtained using a phosphorimager (Molecular Dynamics) using a pixel size of 100 u during the scanning. Images were converted to “TIF” files to allow analysis using Pathways™2.01 software (Research Genetics) and spot intensities compared to indicate changes in RNA expression. Each array contains 3 separate spots of filaggrin DNA and the average of all 3 spot intensities was calculated for each array. Intensities were normalised against the average intensity of all spots on the array.

Results

Filaggrin is well recognised as a marker of epidermal differentiation and increased levels of filaggrin are widely associated with improved barrier function within the skin. Here (table 2) we show that treatment of epidermal cultures with cis-guggul sterone results in elevation of filaggrin mRNA levels, representing a route to the improvement of barrier function within the epidermis.

TABLE 2
Showing data illustrating the influence of
guggul sterone on the expression of
filaggrin in epidermal cultures.

| | Spot | Standard | Changes in the expression |
|---|---|---|---|
| Treatment | intensity | error | relative to vehicle |
| Vehicle | 1865 | 119 | — |
| Cis-guggul sterone | 2885 | 447 | 1.54 (+/−0.17) |

1. A cosmetic method of providing at least one skin care benefit selected from the group consisting of; treating/preventing dry skin; soothing irritated, red and/or sensitive skin; boosting/maintaining involucrin levels; reducing rate of ageing; the method comprising applying to the skin a topical composition, or dosing a systemic composition, said topical or systemic composition comprising an LXRα activating compound according to any one of formula: 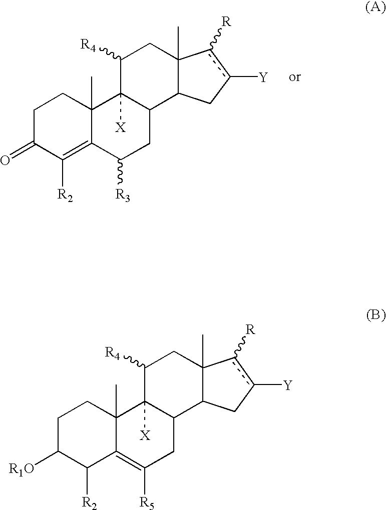
wherein:
R represents a hydrogen, a hydroxyl, a keto, an acetyl, a C₁ to C₇, substituted or unsubstituted, branched or unbranched, saturated or unsaturated alkyl group, or a substituted or unsubstituted, branched or unbranched, unsaturated C₈ alkyl group;
R₁ represents a lower alkyl group, a hydrogen or COR₈;
R₂ represents a hydrogen, a halogen or hydroxyl group;
R₃ represents a hydrogen, a hydroxyl, a halogen, a keto or lower alkyl group;
R₄ represents a hydrogen, a hydroxyl, or a keto group;
R₅ represents a hydrogen, a hydroxyl, a halogen or lower alkyl group;
R₆ represents a lower alkyl group.
X represents a hydrogen, a methyl or a halogen;
Y represents a hydrogen, a hydroxyl, a acetyl or a keto group.

2. The cosmetic method of providing at least one skin care benefit according to claim 2, wherein said LXRα activating compound is present at a level of from 0.001 to 10% wt of the composition.

3. A topical composition for enhancing epidermal barrier function of skin comprising an LXRα activating compound of claim 1 and a dermatologically acceptable vehicle, wherein the R represents —H, —OH, ═O, —COCH₃, —COHCH₃, ═CHCH₂OH, or —OCOCH₃.

4. A systemic composition for enhancing epidermal barrier function of skin comprising an LXRα activating compound of claim 1 and a pharmacologically acceptable carrier therefor, wherein the R represents —H, —OH, ═O, —COCH₃, —COHCH₃, ═CHCH₂OH, or —OCOCH₃.

5. A topical composition for enhancing epidermal barrier function of skin, said composition comprising;
(a an LXRα activating compound selected from the group consisting of, 4-androsten-3,16-dione, 4-androsten-3,16-dione, androst-4-ene-3,6,16-trione, 4-androsten-17beta-ol-3,16-dione acetate, 16-ketotestosterone, 3β-acetoxypregna-5,16-dien-20-one, 3β-acetoxypregna-5-en-20-one, 3β-hydroxypregna-5,16-dien-20-one, 3β-hydroxypregna-5-en-20-one, 5,16-dien-pregnane-3,20-diol, 4,16dienpregna-3,20-dione, 4-pregnen-3,16,20-trione, 4,17(20)-pregnadien-11beta,21-diol-3-one, 5,17(20)-pregnadien-3,16-diol-diacetate, 5,17(20)-pregnadien-3,16-diol, 5-pregnen-3beta,16alpha,21-triol-20-one, 24-hydroxychol-4-en-3-one, cholesta-5,24-dien-3β-ol, stigmasta-5,22-dien-3β-ol, cis-guggal sterone and desmosterol, and mixtures thereof.
(b a dermatologically acceptable vehicle.

6. A systemic composition for enhancing epidermal barrier function of skin, said composition comprising;
(a an LXRα activating compound selected from the group consisting of, 4-androsten-3,16-dione, 4-androsten-3,16-dione, androst-4-ene-3,6,16-trione, 4-androsten-17beta-ol-3,16-dione acetate, 16-ketotestosterone, 3β-acetoxypregna-5,16dien-20-one, 3β-acetoxypregna-5-en-20-one, 3β-hydroxypregna-5,16-dien-20-(one, 3β-hydroxypregna-5-en-20-one, 5,16-dien-pregnane-3,20-diol, 4,16-dienpregna-3,20-dione, 4-pregnen-3,16,20-trione, 4,17(20)-pregnadien-11beta,21-diol-3-one, 5,17(20)-pregnadien-3,16-diol-diacetate, 5,17(20)-pregnadien-3,16-diol, 5-pregnen-3beta,16alpha,21-triol-20-one, 24-hydroxychol-4-en-3-one, cholesta-5,24-dien-3β-ol, stigmasta-5,22-dien-3β-ol, cis-guggal sterone and desmosterol, and mixtures thereof.
(b a pharmacologically acceptable carrier therefor.

7. The composition of claim 5, wherein said activator of LXRα is present 0.01 to 10% wt of the composition.

8. The composition of claim 6, wherein said activator of LXRα is present 0.01 to 10% wt of the composition.
